(19) United States
(12) Patent Application Publication
(10) Pub. No.: US 2010/0286593 A1
(43) Pub. Date: Nov. 11, 2010

(54) BALLOON CATHETER WITH CUTTING FEATURES AND METHODS FOR USE

(75) Inventors: Jeffrey A. Krolik, Campbell, CA (US); Juan Domingo, Sunnyvale, CA (US); Gwendolyn Watanabe, Sunnyvale, CA (US)

Correspondence Address: Vista IP Law Group LLP, 2040 MAIN STREET, Suite 710, IRVINE, CA, 92614, US

(73) Assignee: HOTSPUR TECHNOLOGIES, INC., Mountain View, CA (US)

(21) Appl. No.: 12/778,056

(22) Filed: May 11, 2010

Related U.S. Application Data
(60) Provisional application No. 61/215,901, filed on May 11, 2009.

(51) Int. Cl. A61B 17/22; B29C 47/00

(52) U.S. Cl. 604/22; 606/159; 264/209.3

(57) ABSTRACT

Apparatus and methods are provided for treating a lesion within a body lumen using a catheter including a balloon on its distal end that includes a plurality of raised structures that extend outwardly from the balloon membrane. In one embodiment, the raised structures are provided in pairs that are spaced apart from one another about the circumference of the balloon, and each pair includes two raised structures that are spaced relatively closely together compared to the distance the two raised structures are spaced apart from adjacent pairs. Optionally, the membrane between the two raised structures of each pair may be relatively thinner than the membrane between spaced apart pairs such that the distance between the raised structures in each pair increases as the balloon is expanded within a lesion.

### RELATED APPLICATIONS

This application claims benefit of co-pending U.S. provisional application Ser. Nos. 61/215,901, filed May 11, 2009, the entire disclosure of which is expressly incorporated by reference herein.

### FIELD OF THE INVENTION

The present invention relates generally to apparatus for treating stenoses, occlusions, or other lesions within a body lumen of a patient. More particularly, the present invention relates to catheters including a balloon having cutting or other features to enhance treatment of stenoses, occlusions, or other lesions within a body lumen, and to methods for making and using such catheters.

### BACKGROUND

Medical balloons are frequently used in interventional procedures where a vessel with in the body has become constricted. Standard balloons typically have a substantially uniform wall thickness and a smooth exterior surface. For standard balloons to function, they must be inflated to a pressure sufficient to rupture the constricting tissue, leaving an open lumen within the vessel. Alternatives to a standard balloon may be provided that include structures overlying the balloon to create focused pressure on the constricting tissue.

For example, U.S. Pat. No. 5,196,024 discloses a balloon catheter that includes cutting edges mounted to the external surface of the balloon. While the balloon on this catheter may be able to cut into constricting tissue, the balloon may need to be inflated slowly to prevent cutting or otherwise damaging the balloon itself with the cutting edges. In addition, the balloon may be relatively stiff and difficult to navigate due to the cutting edges attached to the balloon surface. Furthermore, the cost of manufacturing such a balloon may be significantly higher than a standard balloon due to the added cost of providing and attaching the cutting edges to the balloon surface.

Accordingly, apparatus and methods for treating lesions within blood vessels, grafts, or other body lumens would be useful.

### SUMMARY

The present invention is directed to apparatus for treating stenoses, occlusions, or other lesions within a body lumen of a patient. More particularly, the present invention is directed to catheters including a balloon having cutting or other features to enhance treatment of stenoses, occlusions, or other lesions within a body lumen, and to methods for making and using such catheters.

In accordance with one embodiment, an apparatus is provided for treating a lesion within a body lumen that includes a tubular member including a proximal end, a distal end sized for introduction into a body lumen, and a lumen extending between the proximal and distal ends; and a balloon on the distal end. The balloon includes a membrane defining an interior communicating with the lumen and a plurality of raised structures integrally molded with the membrane such that the raised structures extend outwardly from the membrane. In one embodiment, the raised structures include outer pointed edges to enhance penetration into surrounding tissue during expansion of the balloon. In addition or alternatively, the raised structures may extend substantially axially along the balloon or may extend helically or circumferentially around the balloon.

In one embodiment, the raised structures may be provided in pairs that are spaced apart from one another about the circumference of the balloon, and each pair includes two raised structures that are spaced relatively closely together compared to the distance the two raised structures are spaced apart from adjacent pairs. Optionally, the membrane between the two raised structures of each pair may be relatively thinner than the membrane between spaced apart pairs.

In accordance with another embodiment, a method is provided for making a balloon catheter including a plurality of cutting features that includes extruding balloon material to form an elongate tube; placing the elongate tube in a mold having a wall defining a cavity having a proximal end, a distal end, and a shape therebetween corresponding to a balloon, the wall including a plurality of recesses extending at least partially between the proximal and distal ends; and heating the mold to form the tube into a balloon having a membrane extending between the proximal and distal ends of the cavity, and a plurality of raised structures formed in the membrane by the recesses.

Optionally, the method may also include attaching the balloon to the distal end of a tubular member such that an interior of the balloon communicates with an inflation lumen of the tubular member.

In accordance with still another embodiment, an apparatus is provided for treating a lesion within a body lumen that includes an elongate member including a proximal end, a distal end sized for introduction into a body lumen; an expandable member on the distal end including a membrane defining an interior communicating with the lumen; and a plurality of pairs of raised structures extending along an outer surface of the expandable member. The pairs of raised structures may be spaced apart from one another about the circumference of the expandable member, and each pair may include at least two raised structures that are spaced relatively closely together compared to the distance the two raised structures are spaced apart from adjacent pairs. Optionally, the membrane between the two raised structures of each pair may be relatively thinner than the membrane between spaced apart pairs.

In one embodiment, the pairs of raised structures may be integrally molded with the membrane. Alternatively, the pairs of raised structures may be attached to the membrane. For example, the raised structures may be provided on strips of material attached to the membrane. Each strip of material may include opposite longitudinal edges, and a raised structure may be formed on each longitudinal edge. The strips of material may be attached to the membrane such that the distance between longitudinal edges of adjacent strips is less than a distance between the opposite longitudinal edges of each strip such that the raised structures of each strip belong to different pairs of raised structures.

In accordance with yet another embodiment, a method is provided for treating a lesion within a body lumen that includes introducing a distal end of a tubular member into the body lumen with an expandable member thereon in a collapsed condition; expanding the expandable member within the lesion to dilate surrounding tissue, the expandable member including pairs of raised structures spaced apart from one another that penetrate into the surrounding tissue; and expanding the expandable member such that the distance between the raised structures in each pair increases to enhance dilation of the surrounding tissue.

These apparatus and methods herein may allow the constricting tissue to be ruptured at a significantly lower inflation pressure, and/or provide predictability and/or uniformity in how the constricting tissue is ruptured.

Other aspects and features of the present invention will become apparent from consideration of the following description taken in conjunction with the accompanying drawings.

### BRIEF DESCRIPTION OF THE DRAWINGS

It will be appreciated that the exemplary apparatus shown in the drawings are not necessarily drawn to scale, with emphasis instead being placed on illustrating the various aspects and features of the illustrated embodiments.

FIG. 1 is a perspective view of an exemplary embodiment of a catheter including a balloon having a plurality of cutting elements formed thereon.

FIG. 2 is a cross-sectional view of the apparatus of FIG. 1, taken along the line 2-2, with the balloon expanded.

FIG. 3 is a cross-sectional view of an alternative embodiment of an expanded balloon that may be provided on the catheter of FIG. 1.

FIG. 4 is a cross-sectional view of another alternative embodiment of an expanded balloon that may be provided on the catheter of FIG. 1.

### DETAILED DESCRIPTION OF THE EXEMPLARY EMBODIMENTS

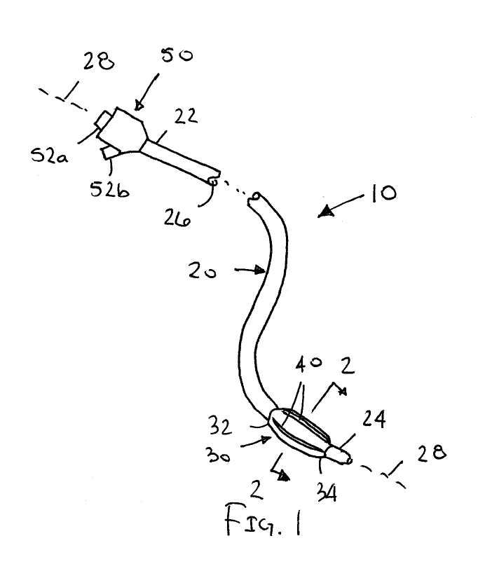

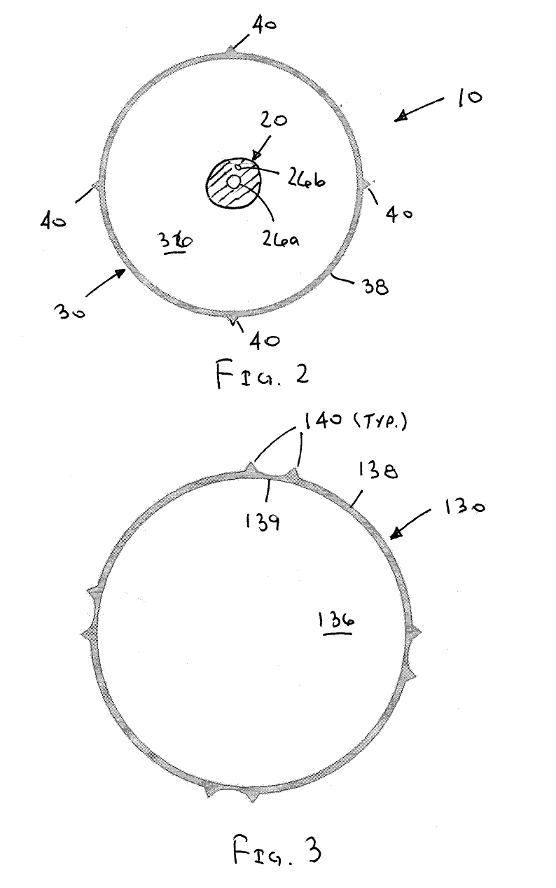

Turning to the drawings, FIGS. 1 and 2 show an exemplary embodiment of an apparatus 10 for treating a body lumen, e.g., for dilating a stenosis, occlusion, or other lesion within the body lumen, such as a blood vessel, aorto-venous fistula, tubular graft, and the like. Generally, the apparatus 10 includes a catheter or other tubular member 20, and a balloon or other expandable member 30 including a plurality of cutting features 40.

The catheter 20 may be an elongate tubular body including a proximal end 22, a distal end 24 sized for introduction into a body lumen, and one or more lumens 26 extending between the proximal and distal ends 22, 24, thereby defining a longitudinal axis 28. The catheter 20 may be configured for introduction and/or advancement through a body lumen, e.g., including a rounded or otherwise substantially atraumatic tip to facilitate advancement into and/or along body lumens within a patient's body, such as the patient's vasculature.

The catheter 20 may have a substantially uniform construction along its length, or alternatively, the construction may be varied. For example, a proximal portion of the catheter 20 may be substantially rigid or semi-rigid to facilitate advancement of the apparatus 10 by pushing or otherwise manipulating the proximal end. In addition or alternatively, a distal portion of the catheter 20 may be flexible, e.g., to facilitate bending and/or advancement through tortuous anatomy without substantial risk of kinking or buckling. In exemplary embodiments, the catheter 20 may be formed from materials such a metal, plastic, e.g., PEEK, Grilamed L25, and the like, or composite materials. The catheter 20 may have a length between about fifty and one hundred fifty centimeters (50-150 cm) and an outer diameter between about one and two millimeters (1.0-2.0 mm).

Optionally, the catheter 20 may include a handle or hub 50 on the proximal end 22. The handle 50 may be shaped to facilitate holding or manipulating the apparatus 10 and/or may include one or more ports 52 communicating with respective lumens 26. For example, a first port 52a may communicate with a first lumen 26a (see FIG. 2) that extends through the catheter 20 for receiving a guidewire, elongate, rail or other instrument (not shown). The handle 50 may include one or more seals, e.g., a hemostatic seal, to provide a substantially fluid-tight seal around a guidewire or other instrument received through the port 26a.

In addition, a second side port 52b may be provided on the handle 50 that communicates with an inflation lumen 26b that extends through the catheter 20 and communicates with an interior 36 of the balloon 30. A source of inflation media and/or vacuum, e.g., a syringe with saline or other fluid (not shown), may be coupled to the side port 52b for delivering inflation media into and/or aspirating inflation media out of the interior 36 for expanded and collapsing the balloon 30. Alternatively, the balloon 30 may be replaced with an expandable member that may be mechanically or otherwise expandable, e.g., including an expandable frame or other structure within or otherwise coupled to a membrane (not shown).

As shown in FIG. 1, the balloon 30 includes proximal and distal ends 32, 34 attached or otherwise coupled to the distal end 26 of the catheter 20 to provide a fluid-tight connection, e.g., by one or more of bonding with adhesive, interference fit, sonic welding, fusing, engagement with a surrounding sleeve or other connector (not shown), and the like. Alternatively, the balloon 30 may include a proximal end 32 attached to the distal end 26 of the catheter 20, and a distal end spaced distally from the distal end 26 and defining an outlet (not shown), e.g., similar to the apparatus disclosed in U.S. Publication No. 2010/0036410, published Feb. 11, 2010, the entire disclosure of which is expressly incorporated by reference herein. In this alternative, the apparatus 10 may include a valve for selectively opening or closing the outlet and/or other features, e.g., similar to the apparatus disclosed in U.S. Publication No. 2010/0036410.

The balloon 30 may be expandable from a low profile, collapsed configuration, e.g., folded or otherwise disposed around or against the outer surface of the catheter 20 to facilitate introduction of the catheter 20 into a patient's body, and a high profile, expanded configuration, e.g., to engage or otherwise contact an inner surface of a body lumen within which the catheter is introduced.

The balloon 30 may be formed from substantially inelastic material, e.g., to provide a non-compliant balloon that expands to a predetermined size when inflated independent of pressure (once a minimum volume and/or pressure is introduced to achieve the predetermined size). Such a non-compliant balloon 30 may expand to the predetermined size even if inflated to relatively high pressures, e.g., until the balloon 30 bursts or otherwise ruptures, e.g., at pressures of ten atmospheres, twenty atmospheres, thirty atmospheres, and the like. Alternatively, the balloon 30 may be formed from elastic material, e.g., to provide a compliant or semi-compliant balloon that may be expanded to a variety of sizes and/or shapes, e.g., based on the amount of fluid and/or pressure within the interior 36 of the balloon 30.

As best seen in FIG. 2, the balloon 30 includes a plurality of raised structures or other cutting features 40 integrally formed with the balloon material, e.g., extending at least partially between the proximal and distal ends 32, 34 of the balloon 30. The cutting features 40 may include a relatively wide base and taper away from the membrane 38 to a pointed edge that extends along the length of the cutting features 40. Thus, the cutting features 40 may have a triangular or other tapered cross-section, e.g., defining a pointed edge that extends continuously and/or uniformly along a length of the cutting features 40. Alternatively, the cutting features 40 may have variable height or other features (not shown) along the pointed edge between ends of the cutting features 40, if desired, e.g., to enhance penetration into tissue.

The cutting features 40 may extend substantially continuously or intermittently between the proximal and distal ends 32, 34, or may extend only partially along the length of the balloon 30. In addition, the cutting features 40 may be spaced apart from one another about the circumference of the balloon 30 and may extend generally axially along the balloon 30, e.g., substantially parallel to the longitudinal axis 28. Alternatively, the cutting features 40 may extend helically, circumferentially, or otherwise around the balloon 30 (not shown).

As shown in FIG. 2, four cutting features 40 are shown that are spaced substantially evenly around the circumference of the balloon 30, although it will be appreciated that the number and spacing of the cutting features 40 may be varied. For example, as few as two or three cutting features may be provided or as many as six, eight, or ten.

As shown in FIG. 2, the balloon 30 is formed from a substantially uniform balloon membrane 38 and the cutting features 40 are raised structures integrally formed with the membrane 38. The membrane 38 and raised structures 40 may be formed from the same material and/or otherwise substantially simultaneously when the balloon 30 is formed.

For example, the balloon 30 may be manufactured by first extruding a tube, and then expanding the tube using pressurized gas inside a heated balloon mold (not shown). To form the raised structures 40, a plurality of slots may be machined or otherwise formed in the wall of the balloon mold to receive the raised structures as the balloon is blown. Thus, the cutting features 40 may be molded into the membrane 38 during the extrusion process, and may not require additional processing or materials to create them. Furthermore, because the cutting features 40 are made from the material used for the balloon membrane 38, the balloon 30 may remain substantially flexible since there are no metallic other dissimilar components added to the balloon 30.

During use, the apparatus 10 may be introduced into a patient's body, e.g., percutaneously into a vessel within the patient's vasculature, such as the femoral or carotid artery, with the balloon 30 in the collapsed configuration. The distal end 24 of the catheter 20 may be advanced through the vasculature until disposed within or adjacent a stenosis, occlusion, or other lesion (not shown) to be treated. Optionally, the lesion may be treated previously, e.g., to remove thrombus or other material before introduction of the apparatus 10, e.g., using apparatus and methods disclosed in U.S. Publications Nos. 2010/0036312 or 2010/0036410, both published Feb. 11, 2010, or International Publications Nos. WO 2009/076482, published Jun. 18, 2009, WO 2010/017537, published Feb. 11, 2010, and WO 2010/034021, published Mar. 25, 2010, the entire disclosures of which are expressly incorporated by reference herein.

With the collapsed balloon 30 disposed within the lesion, a syringe or other source of inflation media (not shown) may be used to introduce fluid through the inflation lumen 26b of the catheter 20 to inflate and expand the balloon 30, thereby dilating the constricting tissue surrounding the lesion. The cutting features 40 may concentrate radially expansive forces on relatively small areas of the surrounding tissue to preferentially rupture the constricting tissue, e.g., at a relatively low balloon inflation pressure compared to a standard balloon. If desired, the balloon 30 may be deflated and the process repeated one or more times after moving the catheter 20, e.g., within the same or another blood vessel. Thereafter, the balloon 30 may be collapsed and the apparatus 10 withdrawn from the patient's body.

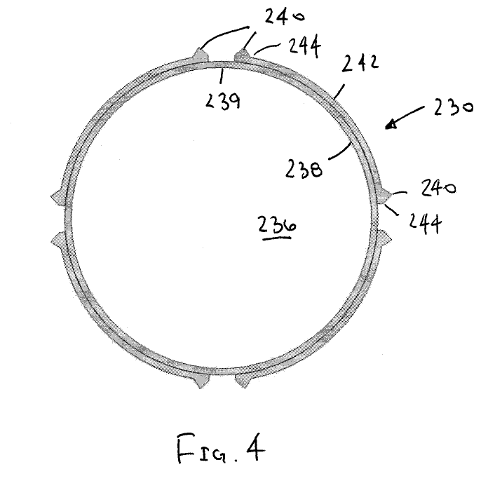

Turning to FIG. 3, an alternative embodiment of a balloon 130 is shown that may be substituted for the balloon 30 on the apparatus 10 of FIG. 1. In this embodiment, a plurality of pairs of cutting features 140 may be provided that are spaced apart about the circumference of the balloon 130. Each pair of cutting features 140 may include two raised structures located in close proximity to each other, and the pairs may be spaced at substantially equal radial distances around the circumference of the balloon 140. Unlike the previous embodiment, the balloon membrane 138 may not have a substantially uniform thickness about its circumference. In particular, the membrane 139 between the cutting features 140 in each pair may be relatively thin compared to the membrane 138 extending between spaced-apart pairs. For example, the membrane 138 may have a wall thickness between about 0.0015-0.0020 inch (0.038-0.051 mm), while the membrane 139 may have a wall thickness between about 0.0005-0.0010 inch (0.013-0.025 mm) or about 0.0005-0.0010 inch (0.013-0.025 mm) thinner than the membrane 138 to provide greater elasticity between the cutting features 140 in each pair, as explained further below.

The cutting structures 140 may function generally similar to the cutting features 40 shown in FIG. 2, e.g., concentrating radial expansive forces on relatively small areas of surrounding tissue to preferentially rupture the constricting tissue at a relatively low balloon inflation pressure. In addition, the pairs of cutting features 140 may provide an additional rupture-inducing force in the constricting tissue—during balloon inflation each pair of cutting features 140 may initially expand radially outwardly into and become mechanically engaged in the constricting tissue. In addition, because of the greater elasticity and/or relative thinner wall of the membrane 139, the distance between the cutting features 140 in each pair may expand preferentially to cause a tensile force in the constricting tissue between the cutting features 140, which may enhance penetration and/or separation of the constricting tissue, which may facilitate dilation of the lesion.

Turning to FIG. 4, another embodiment of a balloon 230 is shown that includes a plurality of pairs of spaced apart cutting features 240 with the region 239 between each pair being relatively thin compared to the overall balloon wall thickness between spaced apart pairs. To provide the balloon 230, a balloon membrane 238 may be formed, e.g., as described above, with a substantially uniform wall thickness. Strips of material 242 may be formed, e.g., from similar or dissimilar material to the membrane 238, that include raised structures or other cutting features 240 formed along opposite side edges 244 thereof. The strips 242 may have lengths corresponding to the length of the balloon 230, e.g., slightly shorter than the balloon 230 if the strips 242 are intended to extend substantially between the proximal and distal ends of the balloon 230.

The strips 242 may be laminated on the outside of the membrane 238, e.g., by bonding with adhesive, fusing, sonic welding, and the like, such that the cutting features 240 extend at least partially along a length of the balloon 230 with the side edges 244 of adjacent strips 242 being spaced apart relatively close to provide each pair of cutting features 240. The resulting balloon 240 thus includes areas where the membrane 238 is overlain with the strips 242 and, consequently, more resistant to stretching than the region 239 between the adjacent cutting features 240. Thus, the region 239 may provide a preferential area of stretch to rupture the constricting tissue similar to the balloon 130 described above.

It will be appreciated that elements or components shown with any embodiment herein are exemplary for the specific embodiment and may be used on or in combination with other embodiments disclosed herein.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

We claim:

1. An apparatus for treating a lesion within a body lumen, comprising:
a tubular member comprising a proximal end, a distal end sized for introduction into a body lumen, and a lumen extending between the proximal and distal ends; and
a balloon on the distal end comprising a membrane defining an interior communicating with the lumen and a plurality of raised structures integrally molded with the membrane such that the raised structures extend outwardly from the membrane.

2. The apparatus of claim 1, wherein the raised structures include outer pointed edges to enhance penetration into surrounding tissue during expansion of the balloon.

3. The apparatus of claim 1, wherein the raised structures extend substantially axially along the balloon.

4. The apparatus of claim 1, wherein the raised structures extend helically around the balloon.

5. The apparatus of claim 1, wherein the raised structures are provided in pairs that are spaced apart from one another about the circumference of the balloon, wherein each pair comprises two raised structures that are spaced relatively closely together compared to the distance the two raised structures are spaced apart from adjacent pairs.

6. The apparatus of claim 5, wherein the membrane between the two raised structures of each pair is relatively thinner than the membrane between spaced apart pairs.

7. A method for making a balloon catheter including a plurality of cutting features, the method comprising:
extruding balloon material to form an elongate tube;
placing the elongate tube in a mold having a wall defining a cavity having a proximal end, a distal end, and a shape therebetween corresponding to a balloon, the wall including a plurality of recesses extending at least partially between the proximal and distal ends; and
heating the mold to form the tube into a balloon having a membrane extending between the proximal and distal ends of the cavity, and a plurality of raised structures formed in the membrane by the recesses.

8. The method of claim 7, further comprising:
providing a tubular member comprising a proximal end, a distal end sized for introduction into a body lumen, and a lumen extending between the proximal and distal ends; and
attaching the balloon to the distal end of the tubular member such that an interior of the balloon communicates with the lumen.

9. An apparatus for treating a lesion within a body lumen, comprising:
a tubular member comprising a proximal end, a distal end sized for introduction into a body lumen, and a lumen extending between the proximal and distal ends;
an expandable member on the distal end comprising a membrane defining an interior communicating with the lumen; and
a plurality of pairs of raised structures extending along an outer surface of the expandable member and spaced apart from one another about the circumference of the expandable member, each pair comprising two raised structures that are spaced relatively closely together compared to the distance the two raised structures are spaced apart from adjacent pairs.

10. The apparatus of claim 9, wherein the membrane between the two raised structures of each pair is relatively thinner than the membrane between spaced apart pairs.

11. The apparatus of claim 9, wherein the pairs of raised structures are integrally molded with the membrane.

12. The apparatus of claim 9, wherein the pairs of raised structures are provided on strips of material attached to the membrane.

13. The apparatus of claim 12, wherein each strip of material includes opposite longitudinal edges, and wherein a raised structure is formed on each longitudinal edge.

14. The apparatus of claim 13, wherein the strips of material are attached to the membrane such that the distance between longitudinal edges of adjacent strips is less than a distance between the opposite longitudinal edges of each strip such that the raised structures of each strip belong to different pairs of raised structures.

15. A method for treating a lesion within a body lumen, comprising:
introducing a distal end of a tubular member into the body lumen with an expandable member thereon in a collapsed condition;
expanding the expandable member within the lesion to dilate surrounding tissue, the expandable member including pairs of raised structures spaced apart from one another that penetrate into the surrounding tissue; and
further expanding the expandable member such that the distance between the raised structures in each pair increases to further enhance dilation of the surrounding tissue.
